(19) United States
(12) Patent Application Publication
(10) Pub. No.: US 2006/0155382 A1
(43) Pub. Date: Jul. 13, 2006

(54) Canulized prosthesis for total hip replacement surgery

(75) Inventor: Scott Steven Katzman, Jupiter, FL (US)

Correspondence Address: STEVEN M. GREENBERG, P.A.;ATTN: STEVEN M. GREENBERG, 12549 EQUINE LANE, WELLINGTON, FL, 33414, US

(73) Assignee: Orthopaedic Development, LLC.

(21) Appl. No.: 11/031,273

(22) Filed: Jan. 7, 2005

(51) Int. Cl. A61F 2/36

(52) U.S. Cl. 623/23.15

(57) ABSTRACT

An orthopedic system including an orthopedic prosthesis having an exterior surface, the prosthesis defining a proximal end and a distal end, a first opening located substantially near the proximal end, a second opening located substantially near the distal end, and a bore extending from the first opening to the second opening. The orthopedic prosthesis also includes a seepage port extending from the exterior surface of the prosthesis and providing a passage to the bore, as well as a guidewire, wherein at least a portion of the guidewire is positionable within the bore, and a resorbable insert positionable within the bore.

### BACKGROUND OF THE INVENTION

1. Statement of the Technical Field

The present invention relates to an orthopedic prosthesis, particularly a system and method for aligning an orthopedic prosthesis.

2. Description of the Related Art

As natural joints in the human body deteriorate due to injury, disease or aging, artificial joint prostheses can be implanted to improve the comfort and quality of life of an individual. Among the more common joint prostheses known are those involved in the replacement of the hip joint. When performing a hip arthroplasty, a cavity is generally created in a proximal portion of a patient's femur which will eventually receive the femoral stem of an implanted prosthesis. Similar replacement techniques for joints other than the hip also include the formation of comparable cavities within existing bone which will eventually house a prosthesis component.

Upon the creation of such a cavity, the prosthesis can be secured using numerous techniques, one of which may include cementing the prosthesis within the cavity. Above all, proper alignment of a prosthetic component is essential if the component is to function correctly. If a prosthesis is misaligned with the femur upon implantation, the misalignment can result in excessive wear of the prosthesis, as well as loosening of the prosthesis within the femoral cavity, and may result in increased pain experienced by the patient. Each of the above consequences may require an additional surgical procedure to repair or realign the prosthesis.

In order to ensure that a prosthetic implant is properly positioned, a surgeon may be required to make a fairly large incision in a patient. However, while a larger incision may provide more room for a surgeon to manipulate the prosthetic implant into proper alignment, it will also result in a larger area of tissue which has to subsequently heal, thereby increasing the size of any scar resulting from the surgical procedure. Consequently, in order to promote healing as well as reduce scarring, a minimally invasive surgical opening is preferable to that of a larger opening. Unfortunately, minimizing the surgical opening reduces the ability of the surgeon to properly position an orthopedic implant. Moreover, lengthy procedures and the use of image guidance equipment are often required to accomplish proper alignment.

In light of the above difficulties, it would be desirable to provide an orthopedic prosthesis which can be properly aligned within a prepared cavity of a bone segment, while minimizing the surgically invasive opening in a patient and in the absence of image guidance equipment.

### SUMMARY OF THE INVENTION

The present invention provides an orthopedic prosthesis which can be properly aligned within a prepared cavity of bone segment, while minimizing the surgically invasive opening in a patient. An exemplary embodiment of the present invention includes an implantable orthopedic prosthesis having an elongate body defining a proximal and a distal end. The implantable orthopedic prosthesis further includes a first opening, a second opening, and a bore extending from the first opening to the second opening. At least one seepage port extends from an exterior surface of the prosthesis to the bore. Moreover, a guide wire is included that is adapted to fit within the bore of the orthopedic prosthesis. Finally, the present invention also includes a resorbable insert containing substrates that promote bone growth, are anti-bacterial, or are anti-inflammatory, where the insert is adapted to fit within the bore of the orthopedic prosthesis.

The present invention further provides a method for implanting an orthopedic device, wherein the guide wire is inserted into an intramedullary canal of a prepared bone. Once the guide wire is positioned, the orthopedic prosthesis is moved along the guide wire, where a portion of the guide wire is located in the bore of the prosthesis. The guide wire is then used to align and steer the prosthesis into the proper position in the prepared bone cavity without the need for image guidance equipment or requiring a large incision. Upon alignment and insertion of the prosthesis, the guide wire is removed, and the resorbable insert is placed in the bore of the orthopedic prosthesis in order to promote healing.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates an orthopedic prosthesis in accordance with the present invention;

FIG. 2 shows a guidewire in accordance with the present invention;

FIG. 3 shows a resorbable insert in accordance with the present invention;

FIG. 4 illustrates an implanted orthopedic prosthesis and a guidewire in accordance with the present invention; and

FIG. 5 shows an implanted prosthesis including a resorbable insert in accordance with the present invention.

### DETAILED DESCRIPTION OF THE INVENTION

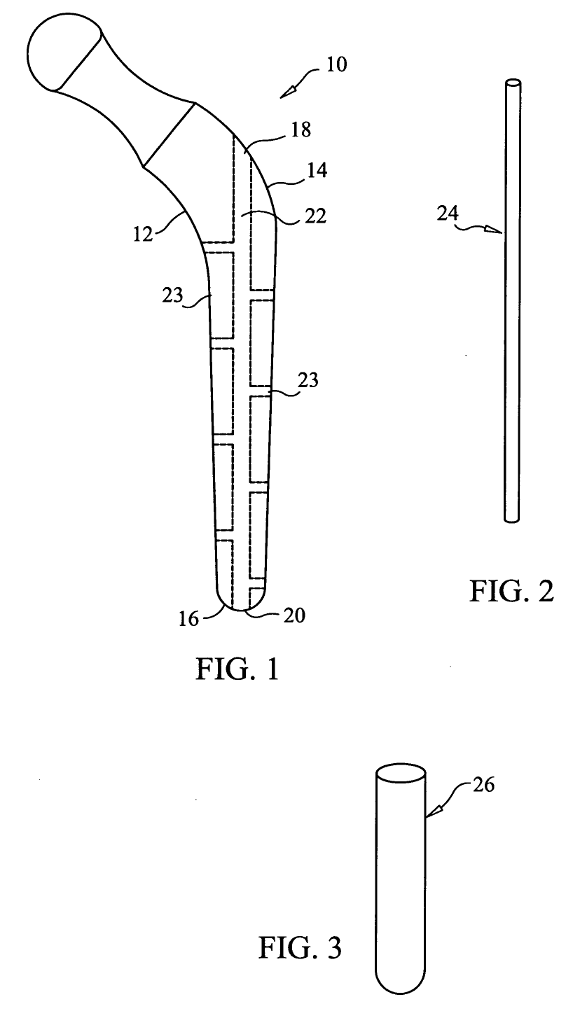

In an exemplary embodiment, the present invention provides an orthopedic system including an orthopedic prosthesis 10 adaptable for implantation into a prepared cavity of a bone, as shown in FIG. 1. The orthopedic prosthesis 10 includes an elongate body 12 defining a proximal end 14 and a distal end 16, and includes a femoral stem prosthesis 12 implanted during a hip arthroplasty procedure. The femoral stem 12 further defines a first opening 18, a second opening 20, and a bore 22 extending from the first opening 18 to the second opening 20. The first opening 18 and the second opening 20 may be located at or substantially near the proximal end 14 and the distal end 16, respectively. However, the location of the first and second openings may be modified in order to facilitate a desired orientation of the prosthesis 10 in a prepared bone cavity upon implantation. By modifying the location of the openings, the alignment of the bore 22 is inherently modified, and may result in the bore 22 not necessarily being aligned or parallel to a central longitudinal axis of the orthopedic prosthesis 10. The bore 22 has a substantially circular cross-section, preferably measuring between 1 mm and 3 mm.

The diameter of bore 22 is preferably kept as small as possible, as having a larger bore diameter would act to reduce the structural integrity of the prosthesis, thus reducing the amount of stress and load that the prosthesis would be able to support. If structural integrity is reduced by a larger diameter bore, there is an increased possibility that the prosthesis will fail under the cyclic loading traditionally experienced by a prosthetic implant when an individual moves. The bore 22 may have a uniform diameter throughout the length of the bore, or, alternatively, may have a decreasing diameter as the bore 22 approaches the second opening 20.

The orthopedic prosthesis optionally may include one or more seepage ports 23 that extend from an exterior surface of the orthopedic prosthesis 10 and providing a passage to the bore 22. The seepage ports 23 have a diameter of less than 1 mm, and provide an in-growth structure so that tissue (such as bone) can grow into the orthopedic prosthesis, thereby enhancing the integration of the orthopedic prosthesis 10 with the surrounding tissue. Although the seepage ports 23 illustrated in FIG. 1 are shown to be substantially horizontal, the angular orientation of the seepage ports 23 may be varied while maintaining the ability of the surrounding tissue and fluid to communicate with the bore 22. Moreover, while the seepage ports 23 are shown to be symmetrically located down the length of the prosthesis 10, the seepage ports may alternatively be staggered on a single side or located non-uniformly in any region of the prosthesis.

Now referring to FIG. 2, the orthopedic system further includes a guide wire 24, where at least a portion of the guide wire 24 is positionable within the bore 22 of the orthopedic prosthesis 10 and the second opening 20, and may further have sufficient length to extend to the end of a reamed femur bone while remaining accessible to the exterior of a surgical opening in a patient. The guide wire 24 has a diameter less than the smallest diameter of the bore 22, and may have a length ranging between 200 mm and 600 mm. The guide wire 24 may be generally constructed from steel, titanium, or medical grade alloy, and further has some flexibility to allow for manipulation and placement of the guide wire 24 into a prepared bone cavity.

A resorbable insert 26 is also included in the orthopedic system 8, as shown in FIG. 3. The resorbable insert 26 is positionable within the bore 22 of the orthopedic prosthesis 10, and may have any length which allows the insert 26 to be completely positioned within the bore 22. The resorbable insert 26 can include numerous substances that promote both bone and tissue healing, as well as prophylactic ingredients such as anti-bacterial agents or anti-inflammatory pharmaceuticals. While by no means an exhaustive list, the resorbable insert 26 can include substances such as bone morphogenic protein, antibiotics, or hormones. Although the resorbable insert 26 is readily absorbed by tissue in contact with the insert at either end of the bore 22 in the orthopedic prosthesis, seepage ports 23 provide increased surface area for tissue and interstitial fluid to come into contact with the resorbable insert, which will increase the rate of absorption of the beneficial substances in the substrate, subsequently providing enhanced healing and recovery.

The orthopedic prosthesis 10 can have a shape adapted to conform to a cavity prepared in a bone, whether substantially circular or rectangular cross-sectional shape. The cross-sectional shape of the prosthesis 10 can be modified in order to adapt to a prepared bone cavity having certain shape characteristics, thus the cross-sectional shape is not limited to a particular orientation.

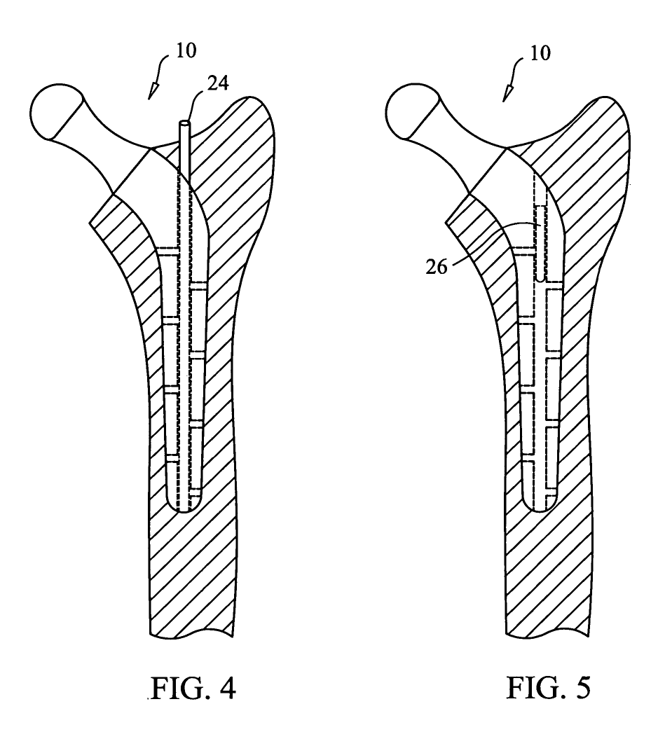

In an exemplary procedure for implanting an orthopedic device, an intramedullary cavity of a bone segment is prepared to receive an orthopedic implant. Such preparation can be carried out by reaming or drilling a cavity within the intramedullary space of a bone. Once the cavity is prepared, at least a portion of the guide wire 24 is inserted into the cavity, with a portion of the guide wire 24 remaining exposed to the exterior of the bone segment. The guide wire 24 may be substantially longer than the length of the bore 22 in order to extend from an extreme end of a reamed femur or bone segment with a portion of the guide wire 24 protruding out of the surgical site. Such extended length allows the manipulation of the guide wire 24 for subsequent alignment of the prosthesis 10. Subsequent to inserting the guide wire 24, the orthopedic prosthesis 10 is positioned over the guide wire 24 such that at least a segment of the exposed portion of the guide wire 24 is positioned within the bore 22 of the orthopedic prosthesis 10. The orthopedic prosthesis 10 is then moved along the guide wire 24 into a desired position within the bone cavity, as shown in FIG. 4. By using the exposed segment of the guide wire 24 as essentially a steering and alignment mechanism, a surgeon is able to position the orthopedic prosthesis 10 in proper alignment with the axis of the bone receiving the implant. Once the orthopedic prosthesis is fully inserted into the prepared cavity, the guide wire 24 is then removed from the orthopedic prosthesis 10, and thus the cavity.

Referring to FIG. 5, upon removal of the guide wire 24, the resorbable insert 26 may then be inserted into the bore 22 of the orthopedic prosthesis. Over time, the resorbable insert may decompose and be absorbed by surrounding tissue, and further, bone and tissue growth may proceed to fill at least a portion of the bore 22 and the seepage ports 23. The tissue and bone growth into the bore 22 may act to promote healing and aid in the integration of the orthopedic prosthesis 10 with the surrounding tissue.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An implantable orthopedic prosthesis, comprising:
a femoral stem having an exterior surface and a central longitudinal axis;
a bore extending from a first opening to a second opening opposite the first opening along the central longitudinal axis; and
at least one seepage port extending from the exterior surface of the femoral stem and providing a passage to the bore inside the femoral stem.

2. The implantable orthopedic prosthesis according to claim 1, wherein the bore has a diameter less than 3 mm.

3. The implantable orthopedic prosthesis according to claim 1, wherein the bore is parallel to the central longitudinal axis of the femoral stem.

4. The implantable orthopedic prosthesis according to claim 1, wherein the seepage port has a diameter less than 1 mm.

5. The implantable orthopedic prosthesis according to claim 1, further comprising a resorbable insert positionable within the bore.

6. The implantable orthopedic prosthesis according to claim 5, wherein the resorbable insert includes an antibacterial substance.

7. The implantable orthopedic prosthesis according to claim 5, wherein the resorbable insert includes a substance that promotes bone growth.

8. The implantable orthopedic prosthesis according to claim 5, wherein the resorbable insert includes an anti-inflammatory substance.

9. An implantable orthopedic prosthesis, comprising:
a femoral stem having an exterior surface and a central longitudinal axis;
a bore extending from a first opening to a second opening opposite the first opening along the central longitudinal axis; and
a plurality of seepage ports extending from the exterior surface of the femoral stem and providing a passage to the bore inside the femoral stem.

10. The implantable orthopedic prosthesis according to claim 9, wherein the plurality of seepage ports are located non-uniformly about the orthopedic prosthesis.

11. The implantable orthopedic prosthesis according to claim 9, wherein the plurality of seepage ports are staggered along a single side of the orthopedic prosthesis.
